(19) United States
(12) Patent Application Publication
(10) Pub. No.: US 2007/0191761 A1
(43) Pub. Date: Aug. 16, 2007

(54) Method of molding for microneedle arrays

(75) Inventors: Mary R. Boone, Naperville, IL (US); Chad J. Carter, Lake Elmo, MN (US); Franklyn L. Frederickson, White Bear Lake, MN (US)

Correspondence Address: 3M INNOVATIVE PROPERTIES COMPANY, PO BOX 33427, ST. PAUL, MN, 55133-3427, US

(73) Assignee: 3M INNOVATIVE PROPERTIES COMPANY, ST. PAUL, MINNESOTA (US)

(21) Appl. No.: 10/590,210

(22) PCT Filed: Feb. 22, 2005
(86) PCT No.: PCT/US05/05466

§ 371 Date: Aug. 22, 2006

Related U.S. Application Data
(60) Provisional application No. 60/546,780, filed on Feb. 23, 2004.

(51) Int. Cl. A61M 37/00; A61B 17/20; A61B 17/06; A61M 5/32; B29C 45/73; C25D 1/00; C25D 7/00; B29C 33/40

(52) U.S. Cl. 604/46; 264/219; 264/220; 264/328.16; 606/223; 604/272; 205/50; 205/67

(57) ABSTRACT

A method of manufacturing a moldable microneedle array (54) is described comprising providing a negative mold insert (44) characterized by a negative image of microneedle topography wherein at least one negative image of a microneedle is characterized by an aspect ratio of between about 2 to 1 and about 5 to 1. The negative mold insert (44) is used to define a structured surface of a negative mold cavity (42). Molten plastic material is injected into the heated negative mold cavity. The molten plastic material is subsequently cooled and detached from the mold insert to provide a molded microneedle array (54). One manner of using microneedle arrays of the present invention is in methods involving the penetration of skin to deliver medicaments or other substances and/or extract blood or tissue through the skin.

This application claims benefit of priority to U. S. Provisional Application Serial No. 60/546,780, filed Feb. 23, 2004.

### FIELD

The present invention relates to the field of methods of manufacturing microneedle arrays.

### BACKGROUND

Only a limited number of molecules with demonstrated therapeutic value can be transported through the skin, even with the use of approved chemical enhancers. The main barrier to the transport of molecules through the skin is the stratum corneum (the outermost layer of the skin).

Devices including arrays of relatively small structures are sometimes referred to as microneedles, microneedle arrays, micro arrays, or micro-pins or the like. These structures have been disclosed for use in connection with the delivery of therapeutic agents and other substances through the skin and other surfaces. These medical devices pierce the stratum corneum by a plurality of microscopic slits in the outermost layer of skin to facilitate the transdermal delivery of therapeutic agents or the sampling of fluids through the skin. The devices are typically pressed or abraded against the skin in an effort to pierce the stratum corneum such that the therapeutic agents and other substances can pass through that layer and into the tissues below.

The vast majority of known microneedle devices include structures having a capillary or passageway formed through the needle. Because the needles are small, the passageways formed in the needles must be limited in size. As a result, the passageways of the needles can be difficult to manufacture because of their small size. There is also a need for the ability to determine the accurate location of the passageways within the needles. A need exists for a method of manufacture for a reduced-cycle time and contaminate-free microneedle array.

Issues associated with microneedle devices include the ability to make precise arrays having microstructured features using biologically acceptable materials. Microneedle arrays have typically been prepared by photoresist manufacturing methods involving the deposition and etching of silicon.

### SUMMARY OF THE INVENTION

The present invention provides methods of molding microneedle arrays. In one embodiment, the microneedle array is manufactured by providing a negative mold insert characterized by the negative image of microneedle topography wherein at least one negative image of a microneedle is characterized by an aspect ratio of between about 2 to 1 and about 5 to 1. The negative moldinsert is transferred into an injection molding apparatus to define a structured surface of a negative mold cavity. The temperature of the negative mold cavity is raised above the softening temperature of the moldable plastic material. In one embodiment, the temperature of the negative mold cavity is raised about 10° C. above the softening temperature of the moldable plastic material. The moldable plastic material is heated to at least the molten temperature of the moldable plastic material in a chamber separate from the negative mold cavity. The molten plastic material is then injected into the heated negative mold cavity and allowed to fill at least about 90 percent of the volume of the negative indentations defined by the negative mold insert. The negative mold cavity is cooled to a temperature at least below the softening temperature of the moldable plastic material and the molded microneedle array or positive mold member is detached from the negative mold insert. In one embodiment, this allows the microreplicated part to be separated from the negative mold insert without distortion.

The present invention also provides methods of manufacturing a negative mold insert used for the preparation of the molded microneedle arrays. In one embodiment, the negative mold insert is manufactured by providing a positive mold master member characterized by microneedle topography wherein at least one microneedle is characterized by an aspect ratio of between about 2 to 1 and about 5 to 1. A negative mold insert is electroformed around the positive mold master and detached from the positive mold master member. Finally, the present invention provides the ability to meet the need for high-volume consistent arrays suitable for use in medical applications.

The features and advantages of the present invention will be understood upon consideration of the detailed description of the preferred embodiment as well as the appended claims. These and other features and advantages of the invention may be described below in connection with various illustrative embodiments of the invention. The above summary of the present invention is not intended to describe each disclosed embodiment or every implementation of the present invention. The Figures and the detailed description which follow more particularly exemplify illustrative embodiments.

### BRIEF DESCRIPTION OF THE DRAWINGS

In the description of the preferred embodiment, reference is made to the various Figures, wherein:

FIGS. 1A-D are schematic diagrams of one manufacturing process for fabricating microneedle arrays in accordance with the methods of the present invention;

FIG. 2 is schematic diagram of one portion of an injection molding apparatus used in accordance with the methods of the present invention;

FIG. 3 is a photomicrograph of an microneedle array according to the present invention;

FIG. 4 is a perspective view of a microneedle array according to the invention;

FIG. 5A is a schematic cross-sectional view of a detailed view of one embodiment of a mold apparatus; and

FIG. 5B is a schematic cross-sectional side view of a detailed view in FIG. 5A.

### DETAILED DESCRIPTION OF ILLUSTRATIVE EMBODIMENTS OF THE INVENTION

The present invention provides a method of manufacturing microneedle arrays that may be useful for a variety of purposes. For example, the microneedle arrays may be used to deliver drugs or other pharmacological agents through the skin utilizing various transdermal drug delivery methods. Alternatively, the microneedle arrays may be used to deliver compounds to the skin or intradermally, such as in the case of vaccines or dermatologic treatments. The microneedles preferably have a size and shape that allow them to penetrate through the stratum corneum or outermost layer of the skin. Where the microneedles are to be used for transdermal drug delivery, the shape and size of the microneedles is preferably sufficient to allow the stratum corneum to be breached. It may be preferable for the microneedles to be sized such that they penetrate into the epidermis. It is also, however, preferable that the size and shape of the microneedles is such that they avoid contact with nerves and the corresponding potential for causing pain when applied to a patient.

In addition to transdermal or intradermal drug delivery, the microneedle arrays of the present invention may also find use as a mechanical attachment mechanism useful for attaching the microneedles arrays to a variety of surfaces. For example, the microneedle arrays may be used to affix a tape or other medical device to, e.g., the skin of a patient.

As used herein, certain terms will be understood to have the meanings set forth below:

“Negative mold cavity” refers to the area in the mold that produces the final part geometry. The negative mold cavity comprises at least one structured surface defined by a negative mold insert having the female or negative structure of the final microneedle array. For example, the negative mold insert may comprise a nickel material that was separated from the positive mold master member which houses pyramidal indentations in the shape of the desired microneedles. These pyramidal indentations protrude into the nickel material from the surface and provide the features that allow the microneedle array to be molded.

“Positive mold master” or “positive mold master member” refers to a tool master having the actual microreplicated geometry of the microneedle array (e.g., pyramidal needles). The positive mold master is used to produce the negative mold insert.

“Negative mold insert” refers to a component of the mold insert apparatus and is formed as the negative image of the positive mold master. The negative mold insert defines one surface of the negative mold cavity and contains the negative image of the microneedle array to be molded

“Array” refers to the medical devices described herein that include one or more microstructures (e.g., pyramidal needles) capable of piercing the stratum corneum to facilitate the transdermal delivery of therapeutic agents or the sampling of fluids through the skin. The array may optionally contain additional non-microstructured features, such as flanges, connectors, etc.

“Microstructure” refers to the specific microscopic structures associated with the array that are capable of piercing the stratum corneum to facilitate the transdermal delivery of therapeutic agents or the sampling of fluids through the skin. By way of example, microstructures can include needle or needle-like structures as well as other structures, such as blades or pins, capable of piercing the stratum corneum. The microstructure is also referred to as a “microneedle”, “micro array” or “microneedle array”.

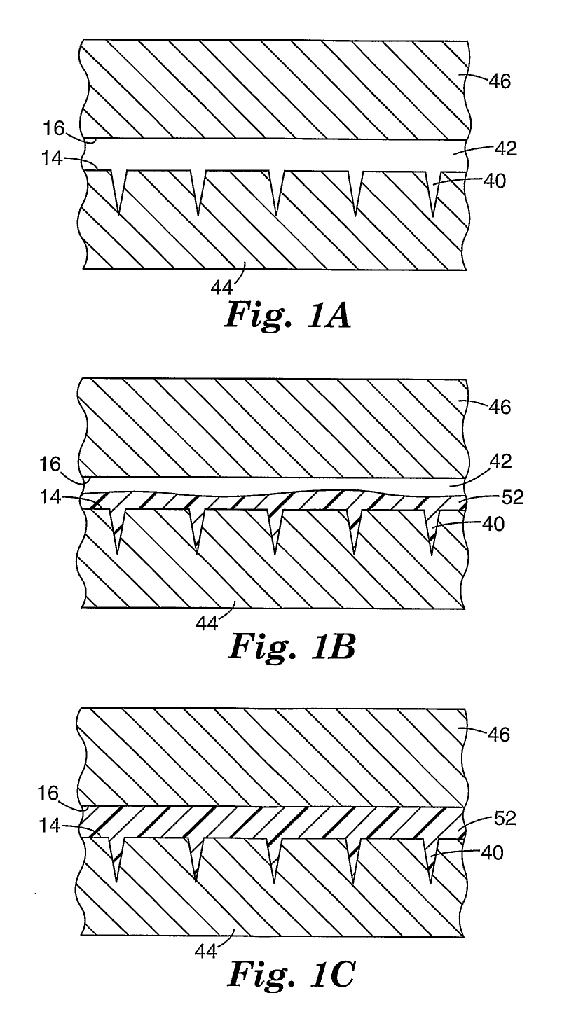

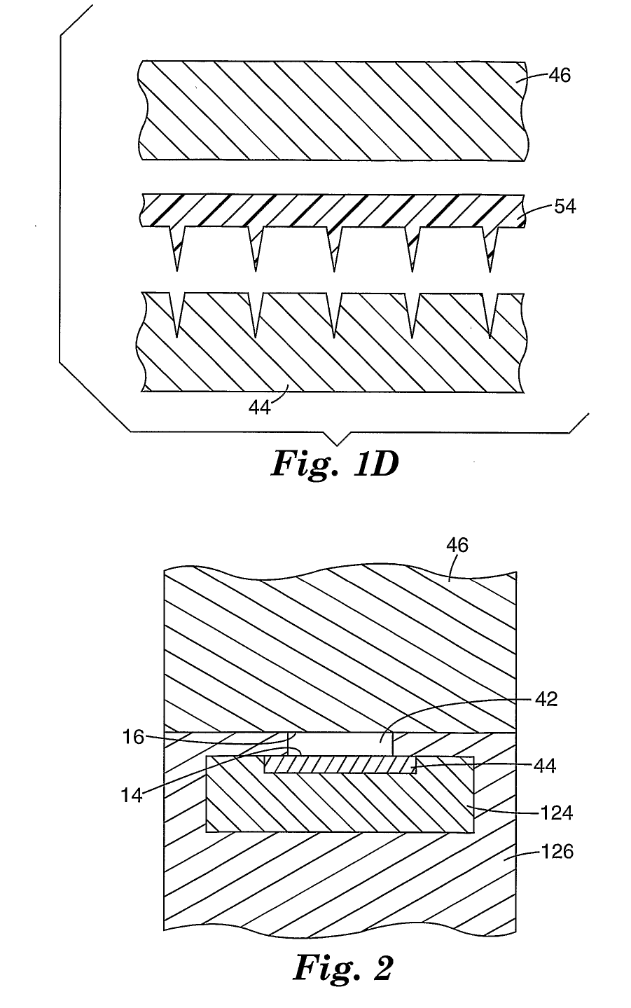

One embodiment for forming microneedle arrays according to the present invention is illustrated in FIGS. 1A-D. Briefly, as shown in FIG. 1A, the method involves providing a negative mold insert 44 which defines a structured surface 14 of a negative mold cavity 42. The opposing surface 16 of the negative mold cavity 42 is defined by a mold apparatus member 46. The structured surface 14 includes cavities 40 having the shape of the desired microneedles and any other features. As depicted the opposing surface 16 is a non-structured or planar surface. In an alternate embodiment, the opposing surface 16 may contain both positive and negative structural features, such as grooves, slots, pins, and needles.

The negative mold insert 44 may be prepared by an electroforming process around a positive mold master (not shown). The process of electroforming involves placing the positive mold master into an electroforming tank that deposits a metal around the features of the master. This may be any suitable metal including, for example, nickel. The nickel is deposited to a desired thickness at which point the positive mold master is separated from the electroformed metal creating the negative mold master or insert for the desired microneedle array. This mold is typically called the electroform. The electroform is then cut to the desired shape to fit into the injection molding apparatus.

Alternatively, the negative mold insert 44 may be prepared directly by laser ablation of a mold substrate (using, e.g., an excimer laser) to provide cavities in the shape of the desired microneedles. Cavities may also be formed by conventional photolithography, chemical etching, ion beam etching, or any other conventional processes known in the art.

As shown in FIG. 1B, the negative mold cavity 42 is then heated to a temperature of more than about 10° C. above the softening temperature of a moldable plastic material. The moldable plastic material is also heated to at least the molten temperature of the moldable plastic material in a chamber (not shown) separate from the negative mold cavity. The molten plastic material 52 is injected into the heated negative mold cavity 42. As depicted, the molten plastic material 52 has partially filled the negative mold cavity 42. It should be understood that FIG. 1B is schematic in nature and that the molten material present in a partially filled mold cavity may be present along either or both surfaces 14 and 16, and further that it may fill (e.g., as a plug) from one side of the mold to the other. The negative mold cavity 42 may be heated using an oil heating system which can be used to control the temperature of the negative mold insert 44 and the mold apparatus member 46. The molten plastic material preferably fills at least about 90 percent,and more preferably at least about 95 percent, of the volume of the cavities 40 defined by the negative mold insert 44. In one embodiment, the molten plastic material fills substantially the entire volume of the cavities 40 defined by the negative mold insert 44, as shown in FIG. 1C. The filled negative mold cavity 42 is then cooled to a temperature at least below the softening temperature of said moldable plastic material. Finally, the molded microneedle array or positive mold member 54 is detached from the negative mold insert 44 and the mold apparatus 46, as shown in FIG. 1D.

Preferably, the molded microneedle array comprises a plurality of molded microneedles having a height greater than about 90 percent of the corresponding height of the microneedle topography in the negative mold insert. More preferably, the molded microneedle array comprises a plurality of molded microneedles having a height greater than about 95 percent of the corresponding height of the microneedle topography in the negative mold insert. It is most preferable that the molded microneedle array comprises a plurality of molded microneedles having a height substantially the same (e.g., 95 percent to 105 percent) as the corresponding height of the microneedle topography in the negative mold insert. The heating of the negative mold insert above the softening temperature of the plastic material allows the plastic material to substantially fill the narrow channels in the negative mold insert that form the negative image of a microneedle array. It is important that the plastic material not be allowed to substantially cool before filling the narrow channels, since it can “skin over” or solidify in the channel prior to complete filling and block further flow of molten material.

The “softening temperature” refers to the temperature at which a plastic material will soften and deform when subject to ordinary forces, such as those encountered during detachment of a molded part from a mold insert. This may be conveniently measured by the Vicat softening temperature, which measures the temperature at which a flat-ended needle penetrates into a test sample (under conditions, for example, of a 50 N loading on the needle and a rate of temperature increase of 120° C./h as described in ASTM D1525-00). For amorphous materials, the softening temperature will be governed by the glass transition of the material, and in some instances the glass transition temperature will be essentially equivalent to the Vicat softening temperature. The glass transition temperature may be measured by methods known to one skilled in the art, such as by differential scanning calorimetry using a typical scanning rate of 10° C./min. Suitable materials include all thermoplastics and thermoset polymers such as polystyrene, polyvinyl chloride, polymethylmethacrylate, acrylonitrile-butadiene styrene, and polycarbonate. For compositions comprising both crystalline and amorphous materials where the bulk properties of the composition are governed by the crystalline material, the softening temperature is governed by the melting of the material and may be characterized by Vicat softening temperature. Examples of such materials include, polypropylene, polybutylene terephthalate, polystyrene, polyethylene, polythermide, polyethylene terephthalate, and blends thereof.

In one embodiment, the negative mold cavity 42 is heated to a temperature of more than about 20° C. above the softening temperature of a moldable plastic material prior to injection of the molten plastic material. In another embodiment, the negative mold cavity 42 heated to a temperature of more than about 30° C. above the softening temperature of a moldable plastic material prior to injection of the molten plastic material.

In one embodiment, the negative mold cavity 42 is cooled to a temperature of less than about 5° C. below the softening temperature of the moldable plastic material prior to detaching the molded microneedle array or positive mold member 54 from the negative mold insert 44. In another embodiment, the negative mold cavity 42 is cooled to a temperature of less than about 10° C. below the softening temperature of the moldable plastic material prior to detaching the molded microneedle array or positive mold member 54 from the negative mold insert 44.

FIG. 2 illustrates a detailed view of the portion of the injection molding apparatus defining a negative mold cavity 42. A structured surface 14 of the negative mold cavity 42 is defined by the negative mold insert 44. The opposed surface 16 of the negative mold cavity 42 is defined by the mold apparatus 46. A mold insert support block 124 facilitates heat transfer to the negative mold insert 44 during the thermocycling process. A mold insert frame 126 defines the sidewalls of the negative mold cavity 42 and holds the mold insert support block 124 and the negative mold insert 44 in place.

In one embodiment, a positive mold master member is used to form the negative mold insert. The positive mold master member is made by forming a material into a shape in which the microneedle array will be molded. This master can be machined from materials that include, but are not limited to, copper, steel, aluminum, brass, and other heavy metals. The master can also be made from thermoplastic or thermoset polymers that are compression formed using silicone molds. The master is fabricated to directly replicate the microneedle array that is desired. The positive mold master may be prepared by a number of methods, including diamond turning of a metal sheet to form a surface having protrusions with any of a variety of shapes, for example, pyramids, cones, or pins. The protrusions of the positive mold master are sized and spaced appropriately, such that the microneedle arrays formed during molding using the subsequently formed negative mold insert have substantially the same topography as the positive mold master.

In one embodiment, the positive mold master is prepared by direct machining techniques disclosed in U.S. Pat. No. 5,152,917 (Pieper, et al.) and U.S. Pat. No. 6,076,248 (Hoopman, et al.), such as diamond turning. A microneedle array can be formed in a surface of a metal positive mold master, e.g., by use of a diamond turning machine, from which is produced a production tool or negative mold insert having an array of cavity shapes. The metal positive mold master can be manufactured by diamond turning to leave the desired shapes in a metal surface which is amenable to diamond turning, such as aluminum, copper or bronze, and then nickel plating the grooved surface to provide the metal master. A production tool or negative mold insert made of metal can be fabricated from the positive mold master by electroforming. These techniques are further described in U.S. Pat. No. 6,021,559 (Smith).

In one embodiment, the injection of the molten plastic material may be performed in conjunction with a packing or injection pressure used to aid in allowing the molten plastic material to fill the negative mold cavity. In one embodiment, this pressure may be greater than about 6,000 psi. In another embodiment, this pressure may be greater than about 10,000 psi. In yet another embodiment, this pressure may be greater than about 20,000 psi.

In one embodiment, the amount of time between injection of the molten plastic material into the negative mold cavity and detachment of the molded microneedle array (i.e., “cycle time”) is sufficient to allow the negative mold cavity to be substantially filled with molten material and the molten plastic material to be subsequently cooled to a temperature below its softening point. The cycle time is preferably less than about 5 minutes, more preferably less than about 3 minutes, and most preferably less than about 90 seconds.

In one embodiment, it may be desirable to add a compressive force to the molten material in the mold cavity in order to assist in filling the finely detailed cavities of the negative mold insert, such as described in U.S. patent application Ser. No. 60/634319, filed on Dec. 7, 2004 and entitled METHOD OF MOlDING A MICRONEEDLE (Attorney Docket No. 57961US002). Additional details regarding injection-compression molding may be found in U.S. Pat. No. 4,489,033 (Uda et al.), U.S. Pat. No. 4,515,543 (Hamner), and U.S. Pat. No. 6,248,281 (Abe et al.).

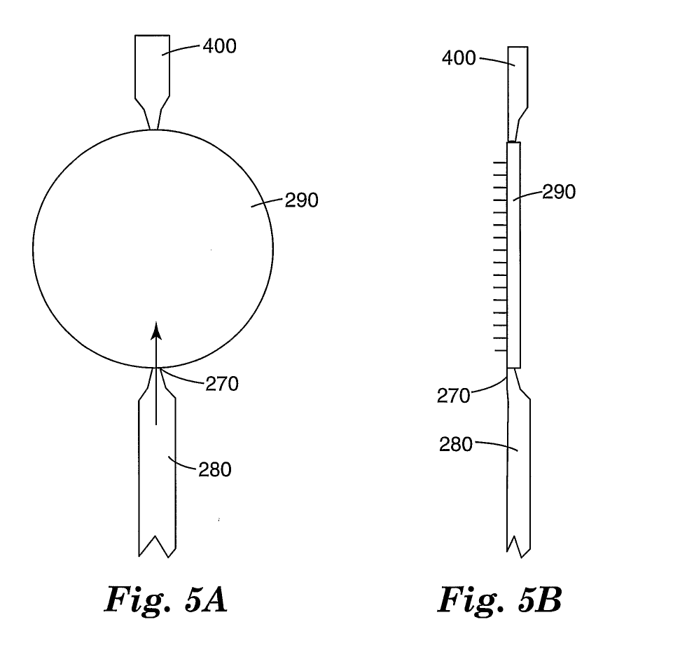

In one embodiment, the molding apparatus includes an overflow vent 400 connected to the negative mold cavity 290, as shown in. FIGS. 5A and 5B. Molten polymeric material fed through the input line 280 passes through the injection gate 270 and into the mold cavity 290. The arrow shows the general direction of flow of polymeric material from the input line 280 into the mold cavity 290. As the polymeric material fills the mold cavity it displaces air that was in the cavity. In one embodiment, little or no displaced air becomes trapped in pockets within the mold cavity or within the negative images of microneedles in the mold insert.

The overflow vent 400 serves as an exit gate to allow displaced air to leave the cavity thus allowing for more uniform filling of the mold cavity with polymeric material. The overflow vent may be positioned anywhere on the outer surface of the mold cavity. In one embodiment the overflow vent is positioned along the sidewalls of the mold cavity. In the embodiment shown in FIGS. 5A and 5 B, the overflow vent 400 is positioned along the sidewall and opposed to the injection gate 270.

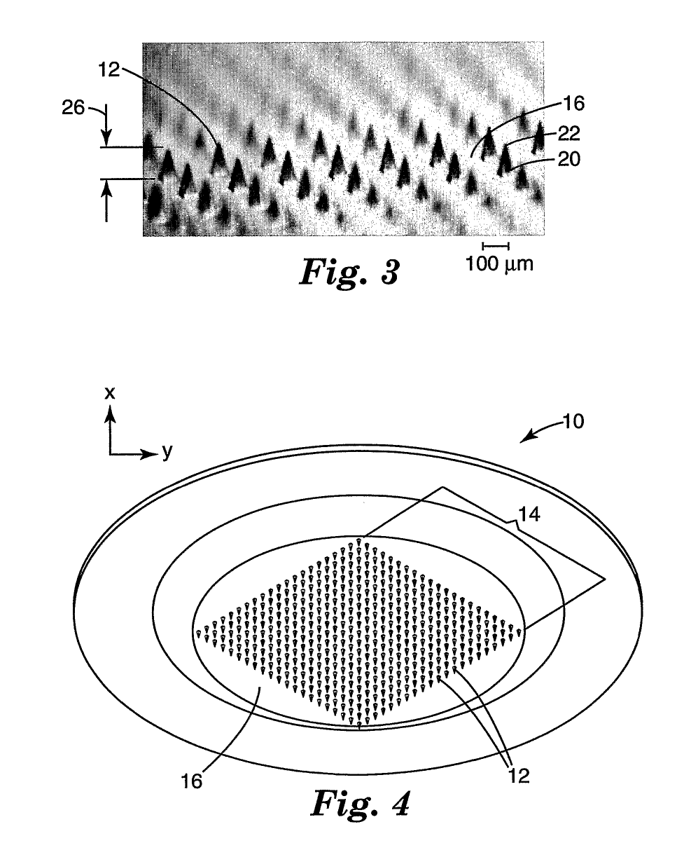

Referring to FIG. 3, each of the microneedles 12 includes a base 20 on the substrate surface 16, with the microneedle terminating above the substrate surface in a tip 22. Although the microneedle base 20 illustrated in FIG. 3 is rectangular in shape, it will be with some bases, e.g., being elongated along one or more directions and others being symmetrical in all directions. The base 20 may be formed in any suitable shape, such as a square, rectangle, or oval. In one embodiment the base 20 may have an oval shape (i.e., that is elongated along an elongation axis on the substrate surface 16).

One manner in which the microneedles of the present invention may be characterized is by height 26. The height 26 of the microneedles 12 may be measured from the substrate surface 16. It may be preferred, for example, that the base-to-tip height of the microneedles 12 be about 500 micrometers or less as measured from the substrate surface 16. Alternatively, it may be preferred that the height 26 of the microneedles 12 is about 250 micrometers or less as measured from the base 20 to the tip 22. It may also be preferred that the height of molded microneedles is greater than about 90%, and more preferably greater than about 95%, of the height of the microneedle topography in the negative mold insert. The microneedles may deform slightly or elongate upon ejection from the negative mold insert. This condition is most pronounced if the molded material has not cooled below its softening temperature, but may still occur even after the material is cooled below its softening temperature. It is preferred that the height of the molded microneedles is less than about 115%, and more preferably less than about 105%, of the height of the microneedle topography in the mold.

The general shape of the microneedles of the present invention is tapered. For example, the microneedles 12 have a larger base 20 at the substrate surface 16 and extend away from the substrate surface 16, tapering to a tip 22. In one embodiment the shape of the microneedles is pyramidal. In another embodiment, the shape of the microneedles is generally conical. In one embodiment the microneedles have a defined tip bluntness, such as that described in co-pending and commonly owned U.S. patent application Serial No. 10/621620, filed on Jul. 17, 2003 and entitled MICRONEEDLE DEVICES AND MICRONEEDLE DELIVERY APPARATUS (Attorney Docket No. 57901US005), wherein the microneedles have a flat tip comprising a surface area measured in a plane aligned with the base of about 20 square micrometers or more and 100 square micrometers or less. In one embodiment, the surface area of the flat tip will be measured as the cross-sectional area measured in a plane aligned with the base, the plane being located at a distance of 0.98 h from the base, where h is the height of the microneedle above the substrate surface measured from base to tip.

The microneedles used in connection with the present invention may have generally vertical wall angles, i.e. the microneedles may be in the form of pins, with sidewalls that are largely orthogonal to the surface of the substrate from which they protrude.

FIG. 4 illustrates a medical device according to one embodiment of the invention in the form of an array 10. A portion of the array 10 is illustrated with microneedles 12 protruding from a microneedle substrate surface 16. The microneedle 12 may be arranged in any desired pattern 14 or distributed over the substrate surface 16 randomly. As shown, the microneedles 12 are arranged in uniformly spaced rows placed in a rectangular arrangement. In one embodiment, arrays of the present invention have a patient-facing surface area of more than about 0.1 cm² and less than about 20 cm² , preferably more than about 0.5 cm² and less than about 5 cm² . In the embodiment shown in FIG. 4, a portion of the substrate surface 16 is non-patterned. In one embodiment the non-patterned surface has an area of more than about 1 percent and less than about 75 percent of the total area of the device surface that faces a skin surface of a patient. In one embodiment the non-patterned surface has an area of more than about 0.10 square inch (0.65 cm₂) to less than about 1 square inch (6.5 cm²). In another embodiment (not shown), the microneedles are disposed over substantially the entire surface area of the array 10.

The microneedle substrates may be manufactured from a variety of materials. Material selection may be based on a variety of factors including the ability of the material to accurately reproduce the desired pattern; the strength and toughness of the material when formed into the microneedles; the compatibility of the material with, for example, human or animal skin; the compatibility of the materials with any fluids that will be expected to contact the microneedle devices, etc.

Among polymeric materials, it is preferred that the microneedles be manufactured of thermoplastic polymeric materials. Suitable polymeric materials for the microneedles of the present invention may include, but are not limited to polyphenyl sulfides, polycarbonates, polypropylenes, acetals, acrylics, polyetherimides, polybutylene terephthalates, polyethylene terephthalates, etc. Polymeric microneedles may be manufactured of a single polymer or a mixture/blend of two or more polymers. In a preferred embodiment the microneedles are formed from polycarbonate. In another preferred embodiment the microneedles are formed from a blend of polycarbonate with polyetherimide. In still another preferred embodiment the microneedles are formed from a blend of polycarbonate with polyethylene terephthalate.

It may be preferred that the polymeric materials have one or more of the following properties: high tensile elongation at break, high impact strength, and high melt-flow index. In one aspect, the melt-flow index as measured by ASTMD1238 (conditions: 300° C., 1.2 kg weight) is greater than about 5 g/10 minutes. The melt-flow index as measured by ASTMD1238 (conditions: 300° C., 1.2 kg weight) is preferably greater than about 10 g/10 minutes, and more preferably between about 20 g/10 minutes and 30 g/10 minutes. In one aspect, the tensile elongation at break as measured by ASTM D 638 (2.0 in/minute) is greater than about 100 percent. In one aspect, the impact strength as measured by ASTM D256, “Notched Izod”, (73° F.) is greater than about 5 ft-lb/inches.

Another manner in which the microneedles of microneedle devices of the present invention may be characterized is based on the aspect ratio of the microneedles. As used herein, the term “aspect ratio” is the ratio of the height of the microneedle (above the surface surrounding the base of the microneedle) to the maximum base dimension, that is, the longest straight-line dimension that the base occupies (on the surface occupied by the base of the microneedle). In the case of a pyramidal microneedle with a rectangular base 20 as seen in FIG. 3, the maximum base dimension would be the diagonal line connecting opposed corners across the base 20. In one embodiment, the microneedles have an aspect ratio greater than or equal to 2:1. In one embodiment, the microneedles have an aspect ratio of about 3:1. In one embodiment, the microneedles have an aspect ratio of between about 2:1 to about 5:1.

The micro arrays useful in the various embodiments of the invention may comprise any of a variety of configurations. Although not depicted, the microneedle devices may include other features such as channels which are described in U.S. Patent Application Publication No. 2003-0045837-A1. The disclosed microstructures in the aforementioned patent application are in the form of microneedles having tapered structures that include at least one channel formed in the outside surface of each microneedle. The microneedles may have bases that are elongated in one direction. The channels in microneedles with elongated bases may extend from one of the ends of the elongated bases towards the tips of the microneedles. The channels formed along the sides of the microneedles may optionally be terminated short of the tips of the microneedles. The microneedle arrays may also include conduit structures formed on the surface of the substrate on which the microneedle array is located. The channels in the microneedles may be in fluid communication with the conduit structures.

Another embodiment for the micro arrays comprises the structures disclosed in U.S. Pat. No. 6,091,975 (Daddona, et al.) which describes blade-like microprotrusions for piercing the skin. Still another embodiment for the micro arrays comprises the structures disclosed in U.S. Pat. No. 6,313,612 (Sherman, et al.) which describes tapered structures having a hollow central channel. Still another embodiment for the micro arrays comprises the structures disclosed in U.S. Pat. No. 6,652,478 (Gartstein, et al.) which describe hollow microneedles having at least one longitudinal blade at the top surface of tip of the microneedle.

Although the illustrative microneedle devices described herein may include multiple microneedles, it will be understood that microneedle devices of the present invention may include only one microneedle on each substrate. Further, although the microneedle devices are all depicted with only one substrate, each device could include multiple substrates, with each substrate including one or more microneedles protruding therefrom. A suitable one-piece construction that includes an array with means for reversibly attaching the array to an applicator is described in the commonly owned pending U.S. Patent Application Ser. No. 60/532987, filed on Dec. 29, 2003, and entitled MEDICAL DEVICES AND KITS INCLUDING SAME (Attorney Docket No. 59402US002).

Microneedle devices of the present invention may have utility for a number of drugs and therapeutic indications. In one aspect, drugs that are of a large molecular weight may be delivered transdermally. It is commonly accepted that increasing molecular weight typically causes a decrease in unassisted or passive transdermal delivery. Microneedle devices of the present invention have utility for the delivery of large molecules that are ordinarily difficult or impossible to deliver by passive transdermal delivery. Examples of such large molecules include proteins, peptides, vaccines, vaccine adjuvants, polysaccharides, such as heparin, and antibiotics, such as ceftriaxone.

In another aspect, microneedle devices of the present invention may have utility for enhancing or allowing transdermal delivery of small molecules that are otherwise difficult or impossible to deliver by passive transdermal delivery. Examples of such molecules include salt forms; ionic molecules, including biphosphonates, such as sodium alendronate or pamedronate; and molecules with physicochemical properties that are not conducive to passive transdermal delivery.

In another aspect, microneedle devices of the present invention may have utility for enhancing or altering transdermal delivery of molecules that may be delivered using passive transdermal delivery, such as nitroglycerin or estradiol. In such cases, the microneedle devices may be used to cause a more rapid onset of delivery or to cause an increased flux when compared to unassisted passive delivery.

In another aspect, microneedle devices of the present invention may have utility for enhancing delivery of molecules to the skin, such as in dermatological treatments or in enhancing immune response of vaccine adjuvants.

The microneedle arrays of the invention may be used in a variety of different manners. One manner of using microneedle arrays of the present invention is in methods involving the penetration of skin to deliver medicaments or other substances and/or extract blood or tissue through the skin. In use, it is generally desirable to provide the microstructures of the array at a height sufficient to penetrate the stratum corneum. When delivering a medicament or therapeutic agent, the agent is typically applied directly to an area of the skin and the array is then applied to the same area of the skin by contacting the skin with the microstructures of the array with sufficient force to puncture the stratum corneum and thereby allow the therapeutic agent to enter the body through the outermost layer of the skin. The parameters for the delivery of therapeutic agents using the medical devices of the invention are suitably described in the aforementioned U.S. Patent Application Publication No. 2003-0045837-A1 and co-pending patent application, Ser. No. 10/621620.

### EXAMPLES

Examples 1-12

Molded microneedle arrays were prepared according to the general procedures described above using a 55-ton injection molding press (Milacon Cincinnati ACT D-Series Injection Molding Press) equipped with a thermocycling unit (Regoplas 301 DG Thermal Cycling Unit). Polycarbonate pellets were loaded into a reciprocating screw and heated until molten. The negative mold insert was heated to a specified temperature (hereafter referred to as the “mold temperature at injection”) above the softening temperature of the material to be molded. The molding cycle was initiated by closing the mold chamber, clamping the mold with 55 tons of force, and injecting a first portion (approx. 50-80% of the part size volume) of the total amount of material from the reciprocating screw into the negative mold insert. The first portion of material was injected into the negative mold insert at a fixed velocity (hereafter referred to as the “injection velocity”). After injecting the first portion of material the process was switched from an injection-driven to a pressure- driven mode by applying a fixed pressure (hereafter referred to as the “pack pressure”) to force the remainder of the molten material into the negative mold insert. The pack pressure was applied for a fixed time (hereafter referred to as the “hold time”). The pack pressure was subsequently released and the negative mold insert was cooled to an ejection temperature (hereafter referred to as the “mold temperature at ejection” which was at or below the softening temperature of the molded material. Then the mold chamber was opened and the part was ejected. Details of the injection velocity, pack pressure, hold time, injection temperature, and ejection temperature used for each example are given in Table 1.

The polycarbonate (Makrolon® 2407, Bayer Polymers) had the following material characteristics: 1) a melt flow index of 20 g/10 minute when measured according to ASTM D1238 at conditions of 300° C. and 1.2 kg;2) a tensile modulus of 350,000 psi (2400 MPa) when measured according to ASTM D638 at a rate of 1 mm/min; 3) a tensile stress at yield of 9400 psi (64 MPa) when measured according to ASTM D638 at a rate of 1 mm/min; 4) a tensile elongation at break of 115% when measured according to ASTM D638 at a rate of 1 mm/min; 5) an Izod notched impact strength of 14 ft-lb/in² (29.4 kJ/m²) when measured according to ASTM D1822, at 73° F. (23° C.); 6) a Vicat softening temperature measured at a rate of 120° C./h of 146° C.

The negative image of the microneedle arrays had the following dimensions. The overall array was square in shape having a diameter of 0.375 inches (0.95 cm). Individual needles on each array were pyramidal in shape with a height of 150 microns and a base side-length of 50 microns, thus giving needles with an aspect ratio of 3:1. The needles were spaced in a regular array with a distance of 200 microns between the tips of adjacent needles. The tips had a truncated tip with a flat top having a side-length of 5 microns. The negative mold insert was configured to produce three arrays, which were contained in one molded part with an overall length of 2.7 inches (6.86 cm), width of 0.82 inches (2.08 cm), and thickness of 0.062 inches (0.157 cm). These arrays were then cut to specific diameters.

The details of the injection velocity, pack pressure, hold time, mold temperature at injection, mold temperature at ejection, and resulting needle height for each example is shown in Table 1. Microneedle height was measured by cutting a cross-section of the resulting arrays and viewing with a stereomicroscope. Measurements were taken as the average of 9 measurements (3 from each individual array).

### Examples C1-C2

Molded microneedle arrays were prepared according to the procedure described in Examples 1-12, with the exception that the mold temperature at injection on each array was reduced to 310° F. (154.4° C.) and 260° F. (126.7° C.) respectively. The comparative examples details of the injection velocity, pack pressure, hold time, mold temperature at injection, mold temperature at ejection, and resulting needle height for each example is shown in Table 1.

TABLE 1

| | Injection | | | Mold | Mold | Average |
|---|---|---|---|---|---|---|
| | Velocity | Pack | Hold | temperature | temperature | needle |
| Example | [inches/sec, | Pressure | time | at injection | at ejection | height |
| Number | (cm/sec)] | [psi, (Mpa)] | [sec] | [° F., (° C.)] | [° F., (° C.)] | [microns] |
| 1 | 0.50 | 12000 | 4 | 340 | 280 | 141 |
| | (1.27) | (81.6) | | (171.1) | (137.8) | |
| 2 | 0.50 | 12000 | 6 | 340 | 280 | 139 |
| | (1.27) | (81.6) | | (171.1) | (137.8) | |
| 3 | 0.50 | 12000 | 2 | 340 | 280 | 134 |
| | (1.27) | (81.6) | | (171.1) | (137.8) | |
| 4 | 0.50 | 8000 | 4 | 340 | 280 | 133 |
| | (1.27) | (54.4) | | (171.1) | (137.8) | |
| 5 | 0.50 | 16000 | 4 | 340 | 280 | 141 |
| | (1.27) | (108.9) | | (171.1) | (137.8) | |
| 6 | 1.50 | 12000 | 4 | 340 | 280 | 139 |
| | (3.81) | (81.6) | | (171.1) | (137.8) | |
| 7 | 0.30 | 12000 | 4 | 340 | 280 | 141 |
| | (0.76) | (81.6) | | (171.1) | (137.8) | |
| 8 | 0.50 | 12000 | 4 | 350 | 280 | 141 |
| | (1.27) | (81.6) | | (176.7) | (137.8) | |
| 9 | 0.50 | 14000 | 4 | 340 | 280 | 155 |
| | (1.27) | (95.3) | | (171.1) | (137.8) | |
| 10 | 0.50 | 14000 | 4 | 350 | 280 | 152 |
| | (1.27) | (95.3) | | (176.7) | (137.8) | |
| 11 | 0.50 | 16000 | 4 | 350 | 280 | 155 |
| | (1.27) | (108.9) | | (176.7) | (137.8) | |
| 12 | 1.50 | 16000 | 4 | 340 | 280 | 155 |
| | (3.81) | (108.9) | | (171.1) | (137.8) | |
| C1 | 0.50 | 12000 | 4 | 310 | 280 | 85 |
| | (1.27) | (81.6) | | (154.4) | (137.8) | |
| C2 | 0.50 | 14000 | 4 | 260 | 260 | 58 |
| | (1.27) | (95.3) | | (126.7) | (126.7) | |

### Examples 13-16

Molded microneedle arrays were prepared according to the procedure described in Examples 1-12, with the exception that individual needles on each array had a height of 375 microns and a base side-length of 125 microns. The needles were spaced in a regular array with a distance of 600 microns between the tips of adjacent needles. Details of the injection velocity, pack pressure, hold time, mold temperature at injection, and mold temperature at ejection used for each example is shown in Table 2.

TABLE 2

| | Injection | | | Mold | Mold | Average |
|---|---|---|---|---|---|---|
| | Velocity | Pack | Hold | temperature | temperature | needle |
| Example | [inches/sec, | Pressure | time | at injection | at ejection | height |
| Number | (cm/sec)] | [psi, (Mpa)] | [sec] | [° F., (° C.)] | [° F., (° C.)] | [microns] |
| 13 | 0.50 | 14000 | 4 | 340 | 280 | 323 |
| | (1.27) | (95.3) | | (171.1) | (137.8) | |
| 14 | 0.50 | 16000 | 4 | 340 | 280 | 322 |
| | (1.27) | (108.9) | | (171.1) | (137.8) | |
| 15 | 0.50 | 16000 | 4 | 360 | 270 | 335 |
| | (1.27) | (108.9) | | (182.2) | (132.2) | |
| 16 | 0.50 | 16000 | 4 | 370 | 270 | 332 |
| | (1.27) | (108.9) | | (187.8) | (132.2) | |

### Examples 17-26

Molded microneedle arrays were prepared according to the procedure described in Examples 1-12, with the exception that the material used was polyetherimide (Ultem ®1010, GE Plastics) having the following material characteristics: 1) a melt flow index of 17.8 g/10 minute when measured according to ASTM D1238 at conditions of 337° C. and 6.6 kg; 2) a tensile modulus of 520,000 psi (3540 MPa) when measured according to ASTM D638 at a rate of 0.2 mm/min; 3) a tensile stress at yield of 16000 psi (110 MPa) when measured according to ASTM D638 at a rate of 0.2 mm/min; 4) a tensile elongation at break of 60% when measured according to ASTM D638 at a rate of 0.2 mm/min; 5) an impact strength of 0.6 ft-lb/in² (1.3 kJ/m²) when measured according to ASTM D256, notched Izod, at 73° (23° C.); 6 ) a Vicat softening temperature measured at a rate of 120° C/h of 219° C. Details of the injection velocity, pack pressure, hold time, mold temperature at injection, and mold temperature at ejection used for each example is given in Table 3.

### Examples C3

Molded microneedle arrays were prepared according to the procedure described in Examples 17-26, with the exception that the mold temperature at injection on the array was reduced to 330° F. (165.6° C.). The mold temperature at ejection was also reduced to 330° F. (165.6° C.). The comparative examples details of the injection velocity, pack pressure, hold time, mold temperature at injection, mold temperature at ejection, and resulting needle height for each example is shown in Table 3.

TABLE 3

| | Injection | | | Mold | Mold | Average |
|---|---|---|---|---|---|---|
| | Velocity | Pack | Hold | temperature | temperature | needle |
| Example | [inches/sec, | Pressure | time | at injection | at ejection | height |
| Number | (cm/sec)] | [psi, (Mpa)] | [sec] | [° F., (° C.)] | [° F., (° C.)] | [microns] |
| 17 | 0.50 | 28000 | 10 | 470 | 400 | 141 |
| | (1.27) | (190.5) | | (243.3) | (204.4) | |
| 18 | 0.50 | 24000 | 10 | 470 | 400 | 145 |
| | (1.27) | (163.3) | | (243.3) | (204.4) | |
| 19 | 0.50 | 24000 | 10 | 490 | 400 | 161 |
| | (1.27) | (163.3) | | (254.4) | (204.4) | |
| 20 | 0.50 | 24000 | 4 | 490 | 400 | 164 |
| | (1.27) | (163.3) | | (254.4) | (204.4) | |
| 21 | 0.50 | 24000 | 4 | 480 | 375 | 146 |
| | (1.27) | (163.3) | | (248.9) | (190.6) | |
| 22 | 1.00 | 24000 | 4 | 480 | 375 | 144 |
| | (2.54) | (163.3) | | (248.9) | (190.6) | |
| 23 | 1.50 | 24000 | 4 | 480 | 375 | 145 |
| | (3.81) | (163.3) | | (248.9) | (190.6) | |
| 24 | 0.50 | 20000 | 4 | 480 | 375 | 147 |
| | (1.27) | (136.1) | | (248.9) | (190.6) | |
| 25 | 0.50 | 24000 | 30 | 480 | 375 | 168 |
| | (1.27) | (163.3) | | (248.9) | (190.6) | |
| 26 | 0.50 | 24000 | 4 | 480 | 350 | 147 |
| | (1.27) | (163.3) | | (248.9) | (176.7) | |
| C3 | 0.50 | 24000 | 4 | 330 | 330 | na |
| | (1.27) | (163.3) | | (165.6) | (165.6) | |

### Examples 27-28

Molded microneedle arrays were prepared according to the procedure described in Examples 1-12, with the exception that the material used was a blend of polyetherimide and polycarbonate (Ultem® ATX200, GE Plastics) having the following material characteristics: 1) a melt flow index of 24 g/10 minute when measured according to ASTM D1238 at conditions of 337° C. and 6.6 kg; 2) a tensile stress at yield of 14000 psi (95 MPa) when measured according to ASTM D638 at a rate of 0.2 mm/min; 3) a tensile elongation at break of 70% when measured according to ASTM D638 at a rate of 0.2 mm/min; 5) an impact strength of 1 ft-lb/in² (2.1 kJ/m2) when measured according to ASTM D256, notched Izod, at 73° F. (23° C.). Details of the injection velocity, pack pressure, hold time, mold temperature at injection, and mold temperature at ejection used for each example is given in Table 4.

TABLE 4

| | Injection | | | Mold | Mold | Average |
|---|---|---|---|---|---|---|
| | Velocity | Pack | Hold | temperature | temperature | needle |
| Example | [inches/sec, | Pressure | time | at injection | at ejection | height |
| Number | (cm/sec)] | [psi, (Mpa)] | [sec] | [° F., (° C.)] | [° F., (° C.)] | [microns] |
| 27 | 0.75 | 16000 | 4 | 450 | 385 | 155 |
| | (1.91) | (108.9) | | (232.2) | (196.1) | |
| 28 | 0.75 | 16000 | 4 | 460 | 380 | 155 |
| | (1.91) | (108.9) | | (237.8) | (193.3) | |

### Example 29

Molded microneedle arrays were prepared according to the procedure described in Examples 1-12, with two exceptions. The pack pressure was set to a first value (21000 psi) for an initial hold time and then lowered to a second value (18000 psi) for a second hold time. In addition, the material used was a blend of polyethylene terephthalate and polycarbonate (Xylex™ X7110, GE Plastics) having the following material characteristics: 1) a melt flow index of 10.5 g/10 minute when measured according to ASTM D1238 at conditions of 300° C. and 1.2 kg; 2) atensile modulus of 237,000 psi (1610 MPa) when measured according to ASTM D638 at a rate of 2.0 mm/min; 3) a tensile stress at yield of 6600 psi (45 MPa) when measured according to ASTM D638 at a rate of 2.0 mm/min; 4) a tensile elongation at break of 150% when measured according to ASTM D 638 at a rate of 2.0 mm/min; 5) an impact strength of 15 ft-lb/in (31.5 kJ/m²) when measured according to ASTM D256, notched Izod, at 73° F. (23° C.) ); 6) a Vicat softening temperature measured at a rate of 120° C./h of 106° C. Details of the injection velocity, pack pressure, hold time, mold temperature at injection, and mold temperature at ejection used for the example is given in Table 5.

TABLE 5

| | Injection | | | Mold | Mold | Average |
|---|---|---|---|---|---|---|
| | Velocity | Pack | Hold | temperature | temperature | needle |
| Example | [inches/sec, | Pressure | time | at injection | at ejection | height |
| Number | (cm/sec)] | [psi, (Mpa)] | [sec] | [° F., (° C.)] | [° F., (° C.)] | [microns] |
| 29 | 0.24 | 21000/18000 | .5/4 | 290 | 195 | 143 |
| | (0.61) | (142.9/122.5) | | (143.3) | (90.6) | |

It will be appreciated by those skilled in the art that the foregoing detailed description is not to be construed as limiting the ultimate manufacture of the medical devices (e.g., the arrays). The described embodiments, while exemplary of structures contemplated as being within the scope of the invention, are not exhaustive. Accordingly, all numbers are assumed to be modified by the term “about.”

All patents, patent applications, and publications cited herein are each inc herein by reference in their entirety, as if individually incorporated by reference. Various modifications and alterations of this invention will become apparent to those skilled in the art without departing from the scope of this invention, and it should be understood that this invention is not to be unduly limited to the illustrative embodiments set forth herein.

1. A method of manufacturing a molded microneedle array comprising:
providing a negative mold insert characterized by a negative image of microneedle topography wherein at least one negative image of a microneedle is characterized by an aspect ratio of between about 2 to 1 and about 5 to 1;
transferring the negative mold insert into an injection molding apparatus, wherein the negative mold insert is exposed and defines a structured surface of a negative mold cavity;
heating the negative mold cavity to a temperature above the softening temperature of a moldable plastic material;
heating the moldable plastic material to at least the molten temperature of the moldable plastic material in a chamber separate from the negative mold cavity;
injecting the molten plastic material into the heated negative mold cavity,
allowing the molten plastic material to fill at least about 90 percent of the volume of the negative indentations defined by the negative mold insert;
cooling the molten plastic material to a temperature below the softening temperature of the moldable plastic material; and
detaching the molded microneedle array from the negative mold insert.

2. A method of manufacturing a molded microneedle array comprising:
providing a negative mold insert characterized by a negative image of microneedle topography wherein at least one negative image of a microneedle is characterized by an aspect ratio of between about 2 to 1 and about 5 to 1;
transferring the negative mold insert into an injection molding apparatus, wherein the negative mold insert is exposed and defines a structured surface of a negative mold cavity;
heating the negative mold cavity to a temperature of more than about 10 degrees centigrade above the softening temperature of a moldable plastic material;
heating the moldable plastic material to at least the molten temperature of the moldable plastic material in a chamber separate from the negative mold cavity;
injecting the molten plastic material into the heated negative mold cavity,
allowing the molten plastic material to fill at least about 90 percent of the volume of the negative indentations defined by the negative mold insert;
cooling the molten plastic material to a temperature at least below the softening temperature of the moldable plastic material; and
detaching the molded microneedle array from the negative mold insert.

3. A method according to claim 1,
wherein the negative mold insert is formed by:
providing a positive mold master member characterized by microneedle topography wherein at least one microneedle is characterized by an aspect ratio of between about 2 to 1 and about 5 to 1;
electroforming a negative mold insert around the positive mold master; and
detaching the negative mold insert from the positive mold master member.

4. A method according to claim 3, wherein the positive mold master member comprises copper.

5. A method according to claim 1, wherein the negative mold insert is fabricated by nickel electroforming.

6. A method according to claim 3, wherein the microneedle topography of the positive mold master member is prepared by diamond turning.

7. A method according to claim 1, wherein the microneedle array comprises,a plurality of microneedles each having a flat tip comprising a surface area measured in a plane aligned with the base of about 20 square micrometers or more and 100 square micrometers or less.

8. A method according to claim 1, wherein the microneedle array is formed as part of a larger array, wherein at least a portion of the larger array comprises a non-patterned substrate.

9. A method according to claim 8, wherein the non-patterned substrate has an area of more than about 0.10 square inch (0.65 cm²) to less than about 1 square inch (6.5 cm ²).

10. A method according to claim 1, wherein the microneedle array comprises a plurality of molded microneedles having a height greater than about 90 percent of the corresponding height of the microneedle topography in the negative mold insert.

11. (canceled)

12. A method according to claim 1, wherein the moldable plastic material comprises a material selected from the group consisting of polycarbonate, polystyrene, polyethylene, polypropylene, and blends thereof.

13-14. (canceled)

15. A method according to claim 2, wherein the negative mold cavity is heated to a temperature of more than about 30 degrees centigrade above the softening temperature of the moldable plastic material.

16. A method according to claim 1, wherein the microneedle array comprises a plurality of microneedles having a pyramidal shape.

17. A method according to claim 1, wherein the molten plastic material is injected into the heated negative mold cavity with a velocity of less than 2.0 in/sec (5.08 cm/sec).

18. A method according to claim 17, wherein after injection of the molten material, it is held at a packed pressure of more than about 6000 psi (40.8 Mpa).

19. A method of manufacturing a negative mold insert used for preparing molded microneedle arrays comprising:
providing a positive mold master member characterized by microneedle topography wherein at least one microneedle is characterized by an aspect ratio of between about 2 to 1 and about 5 to 1;
electroforming a negative mold insert around the positive mold master; and
detaching the negative mold insert from the positive mold master member.

20. A method according to claim 19, wherein the positive mold master member comprises copper.

21. A method according to claim 19, wherein the negative mold insert is fabricated by nickel electroforming.

22. (canceled)

23. A method according to claim 19, wherein the positive mold master comprises a plurality of microneedles each having a flat tip comprising a surface area measured in a plane aligned with the base of about 20 square micrometers or more and 100 square micrometers or less.

24-25. (canceled)

26. A method of manufacturing a molded microneedle array comprising:
providing a negative mold insert prepared according to claim 19,
transferring the negative mold insert into a molding apparatus, wherein the negative mold insert is exposed and defines a structured surface of a negative mold cavity;
providing a heated plastic material into the negative mold cavity,
allowing the heated plastic material to fill at least about 90 percent of the volume of the negative indentations defined by the negative mold insert;
cooling the plastic material to a temperature at least below the softening temperature of the plastic material; and
detaching the molded microneedle array from the negative mold insert.

27. A method according to claim 26, wherein the molding apparatus is an injection molding apparatus.

28. A method according to claim 26, wherein the negative mold cavity is heated to a temperature of more than about 10 degrees centigraigrade above the softening temperature of the plastic material prior to providing the heated plastic material to the negative mold.

29. A product manufactured according to the process of claim 1.

30. A product according to claim 29, wherein the product is a drug delivery device.
